(19) United States
(12) Patent Application Publication
(10) Pub. No.: US 2008/0319365 A1
(43) Pub. Date: Dec. 25, 2008

(54) Adhesive Posture Supports

(76) Inventor: Ryan Joseph Kendrick, New South Wales (AU)

Correspondence Address: KIRTON AND MCCONKIE, 60 EAST SOUTH TEMPLE,, SUITE 1800, SALT LAKE CITY, UT, 84111, US

(21) Appl. No.: 12/096,635

(22) PCT Filed: Dec. 20, 2006
(86) PCT No.: PCT/AU2006/001938

§ 371 Date: Jun. 9, 2008

(30) Foreign Application Priority Data
Dec. 23, 2005 (AU) 2005907279

(51) Int. Cl. A61F 5/00; A61F 13/00

(52) U.S. Cl. 602/19; 602/20; 602/36; 602/60

(57) ABSTRACT

A self adhesive support which conforms to the human back and spans a number of joints thereby maintaining body posture in the range of the neutral spine whether the wearer is sitting, standing or lying and which is made from a material which is flexible enough to adapt to the wearer's body shape but rigid enough to maintain correct body posture such as orthopedic foam, rubber or soft plastics and which is sufficiently inelastic to limit flexure from the neutral spinal position but is sufficiently elastic to assist movement back into the correct posture. The support may be in the shape of an X and applied to the wearer's back. In an alternative form, the support is in the form of the sides of a square and is applied to the base of the wearer's back.

### FIELD OF THE INVENTION

This invention relates to supports which are applied to the human body for musculoskeletal physiotherapy to relieve pain caused by poor posture.

### BACKGROUND OF THE INVENTION

Back pain, neck pain and cervicogenic headaches which arise from the neck and related structures affect most of the population at some stage during their lives. In many circumstances this pain is related to poor habitual posturing. Due to the viscoelastic properties of the body's tissues, assuming the same posture over an extended period leads to a gradual lengthening of the tissues, which in turn can lead directly to pain or to instability and consequent pain or dysfunction. Poor posture may hasten this effect due to the lack of muscular support and the direct load imparted on passive connective tissues for maintenance of spinal stability and posture.

Increased thoracic kyphotic postures and a forward head position have been related to headaches and neck pain. Low back pain has been strongly associated with a deficiency in the deep spinal stabilising musculature consisting of the deep fibres of lumbar multifidus, transversus abdominis, the pelvic floor muscles and the diaphragm. Recent research suggests that adopting an upright sitting posture with a neutral spinal alignment results in an increase in activity of the deep lumbar multifidus muscle when compared to slouched or flat back sitting. Similarly, improved function of the pelvic floor muscles has been associated with upright sitting. Preliminary evidence suggests that this may occur with not only active but also passive correction of sitting posture.

Poor function of the upper cervical flexors (longus capitis and longus colli) has been demonstrated in subjects suffering from cervicogenic headaches yet not those suffering from other headache forms. Slouched posturing with forward head postures is suggestive of poor function of these muscles. As these muscles are at a mechanical disadvantage, they are difficult to activate effectively in such a posture. It follows that maintenance of an upright posture with appropriate head on neck position will allow optimal recruitment of the upper cervical flexors and at the same time reduce load on potentially nociceptive structures.

There are a number of strategies aimed at restoring function in the deep spinal stabilising muscles such as the use of Electromyographic (EMG) and Ultrasound Biofeedback. Also interventions exist to improve posture through the use of lumbar rolls and postural taping and bracing. Braces such as those disclosed in U.S. Pat. No. 5,105,828, EP 1 523 966, WO 1997/49458, WO 2004/049870 and AU 2003270984 are cumbersome, intrusive and therefore hot and uncomfortable due to rubbing. They may also not result in the maintenance of posture in the range of the neutral spinal position since they are not fitted precisely to the individual. Others only serve to maintain good sitting posture but are ineffective in standing.

Taping techniques can assist in maintaining posture and reducing symptoms however they are time consuming and require skill in their application. This does not bode well for home or self-application. Self adhesive shaped body supports are disclosed in U.S. Pat. No. 6,506,957 but there are no anatomical drawings and there is no teaching as to how the supports are to be applied to the human body. Further the specification gives no explanation of the “proprioceptive effect” nor the “intended medical purposes” referred to therein. The self adhesive shaped body disclosed is referred to as a “cushioning pad” which suggests that its intended use is merely to act as a cushion while sitting. Further the teaching is confined to the process of manufacture of the supports and as such offers no solution to problems relating to incorrect posture.

U.S. Pat. No. 5,228,428 has a single claim to a method of relaxation of contracture of paravertebral muscles of a patient comprising affixing by adhesion substantially non elastic horizontal and vertical traction strips generally at the base of the spine. However it does not specify precise positioning and as will be disclosed below, positioning is critical to the efficacy of any adhesive posture support. Similarly U.S. Pat. No. 5,107,862 teaches a single strip support which only partially adheres to the wearer and is applied to restrict gross curvature of the spine. It does not act to support the more normal musculature of the spine to prevent gross curvature in the first instance.

### OBJECT OF THE INVENTION

It is therefore an object of the present invention to provide supports which overcome some of the disadvantages and limitations of prior art supports or at least provide a useful alternative.

### STATEMENT OF THE INVENTION

According to one but not necessarily the only aspect, the invention resides in a support which is self-adhesive, conforms to the human back spanning a number of joints thereby maintaining body posture in the range of the neutral spine whether the wearer is sitting, standing or lying.

Preferably the support is made from a material which is flexible enough to adapt to the wearer's body shape but rigid enough to maintain correct body posture such as orthopaedic foam, rubber or soft plastics.

Preferably the support material is sufficiently inelastic to limit flexure from the neutral spinal position but sufficiently elastic to assist movement back into the correct posture.

In a preferred form the support is in the shape of an X and is applied to the wearer's back between the points of the shoulders and the contralateral, posterolateral trunk in the region of the lower ribs.

Preferably a cross brace section spans the upper arms of the X shaped support reinforcing the upper arms.

An alternative support is in the form of the sides of a square and is applied to the base of the wearer's back with the lower side in the region of the posterior superior iliac spines (PSIS) and the upper side in the region of the thoracolumbar junction thus reducing flexion of the lumbar spine and preserving neutral spinal alignment.

Preferably the self adhesive is applied to the support during manufacture and protected by a peel off strip which is removed prior to application in the case of single use.

Alternatively the support is provided with double sided adhesive strip which is attached prior to application to the wearer and can be reused with new double sided adhesive replacing the old.

Preferably the support is perforated to allow the wearer's skin to breathe and for moisture to escape.

Preferably the corners of the support are rounded for optimal adhesion and wearer comfort.

Preferably the extremities of the arms of the X shaped support are circular enlargements forming discs which deform when tension is applied.

Preferably the external corners of the square shaped support are circular enlargements forming discs which deform when tension is applied.

### BRIEF DESCRIPTION OF THE DRAWINGS

An embodiment of the invention is now described by way of example only with reference to the accompanying drawings in which:

FIG. 1 shows alternative forms of the support according to the present invention in place on the wearer

FIG. 2 shows the protective strip being removed from the adhesive of a support

FIG. 3 shows double sided adhesive being applied to a support

FIG. 4 shows the supports of FIG. 1 being applied to the wearer

FIG. 5 shows adhesive strips on a support

FIG. 6 shows perforations in a support

FIG. 7 is a side on view of the wearer of FIG. 1

FIG. 8 illustrates the relevant spinal anatomy

FIG. 9 shows an X shaped support with circular extremities and

FIG. 10 shows a square shaped support with circular external corners.

### DETAILED DESCRIPTION OF THE INVENTION

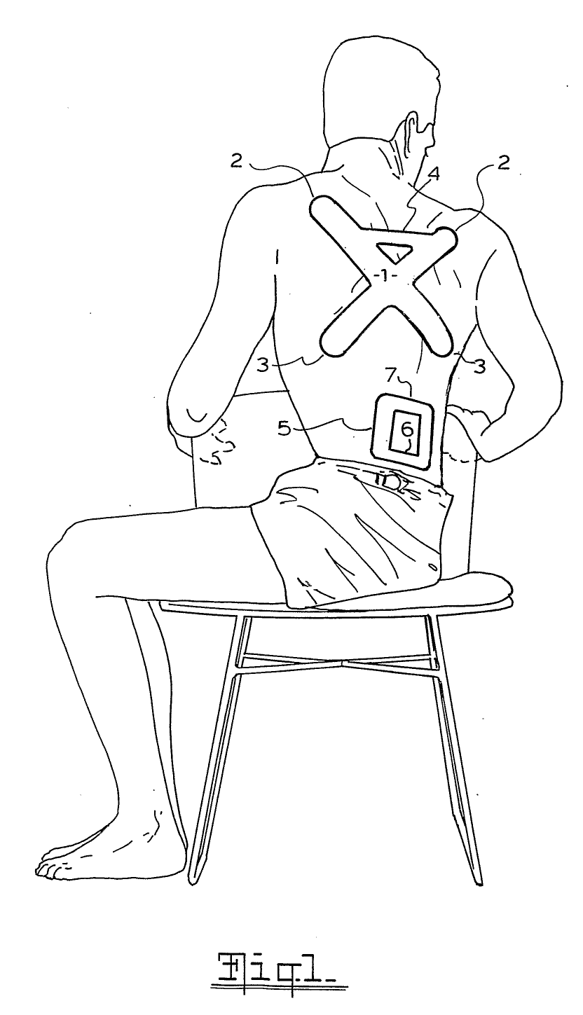

FIG. 1 shows a seated person wearing alternative forms of the support of the present invention. An X shaped support 1 is located on the upper back with the arms of the X reaching between points 2 on the shoulders and the contralateral, posterolateral trunk in the region of the lower ribs 3. A cross brace section 4 spans the upper arms of X shaped support 1 reinforcing its upper arms.

A support 5 in the form of the sides of a square is located at the base of the wearer's back with lower side 6 in the region of the posterior superior iliac spines (PSIS) and the upper side 7 in the region of the thoracolumbar junction. This placement reduces flexure of the lumbar spine and maintains the wearer's neutral spinal alignment.

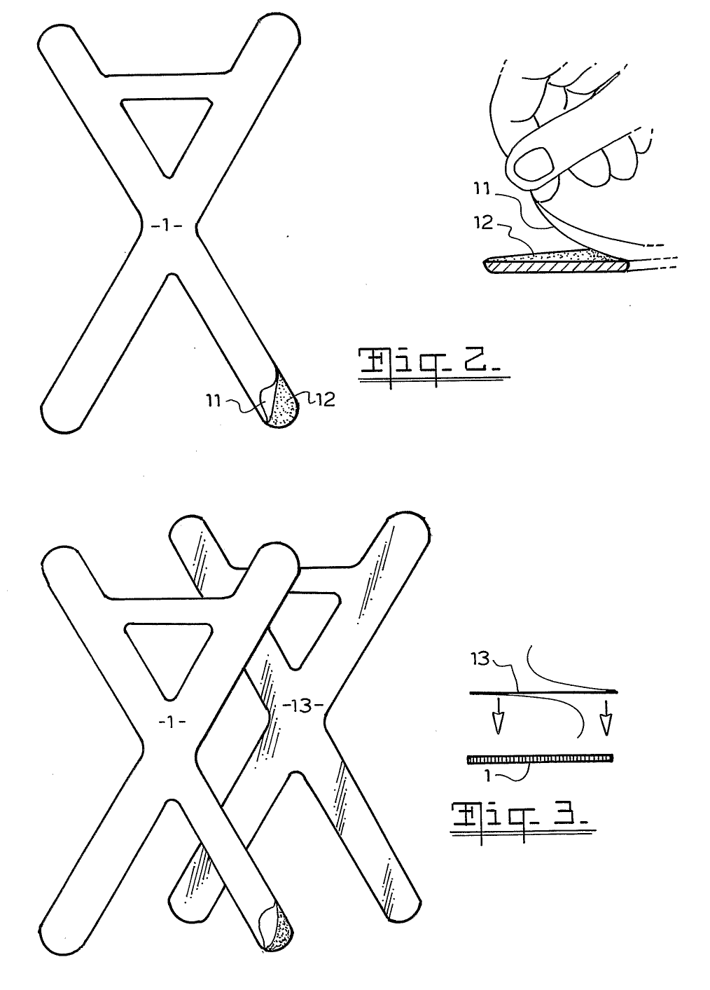

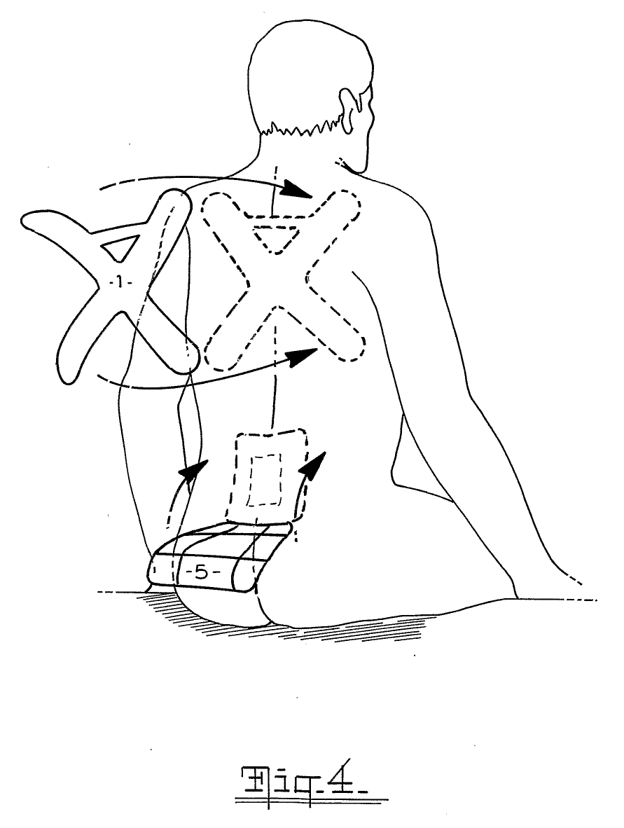

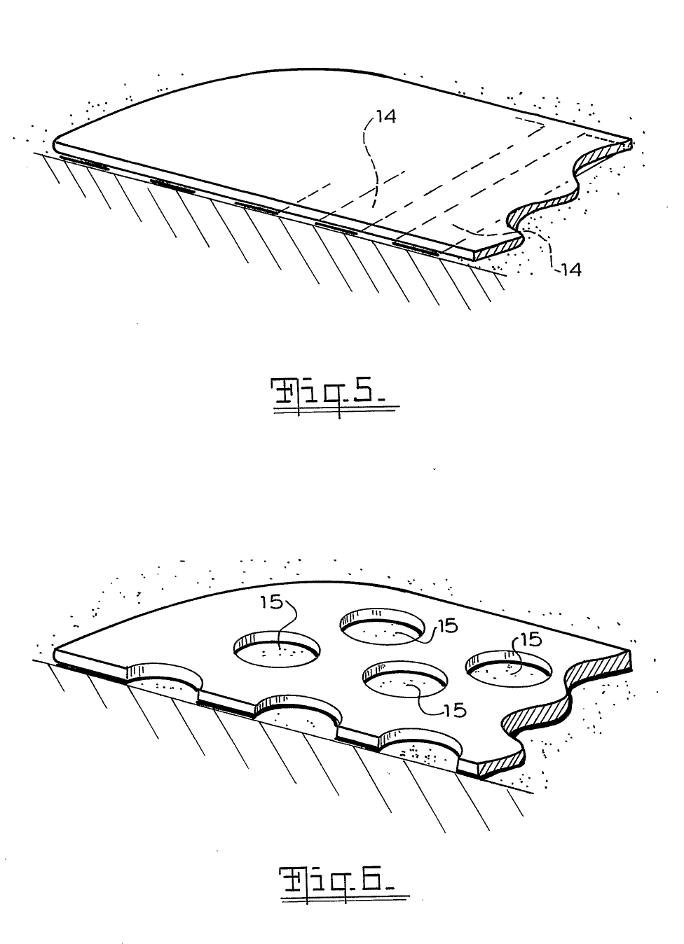

FIG. 2 illustrates support 1 with strip 11 which covers adhesive surface 12 until it is peeled off prior to application to the wearer. This is the single use version of the support but it is also possible to make a multiple use version by providing double sided adhesive 13 as illustrated in FIG. 3. FIG. 4 illustrates the application of supports 1 and 5 to the upper and lower back respectively of the wearer.

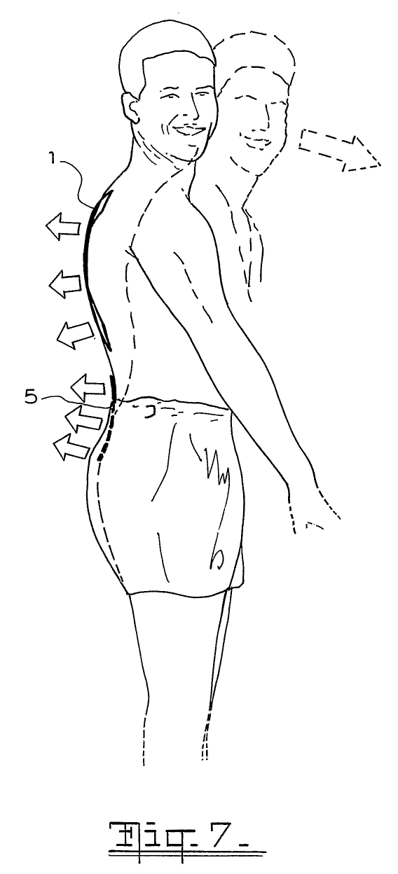

FIG. 5 illustrates a method of applying adhesive to the supports in strips 14 rather than covering the entire support with adhesive. This makes attachment and removal of the support to the wearer easier and less painful. Perforations 15 can also be provided in the support as shown in FIG. 6 which allow the skin to breathe and moisture to escape.

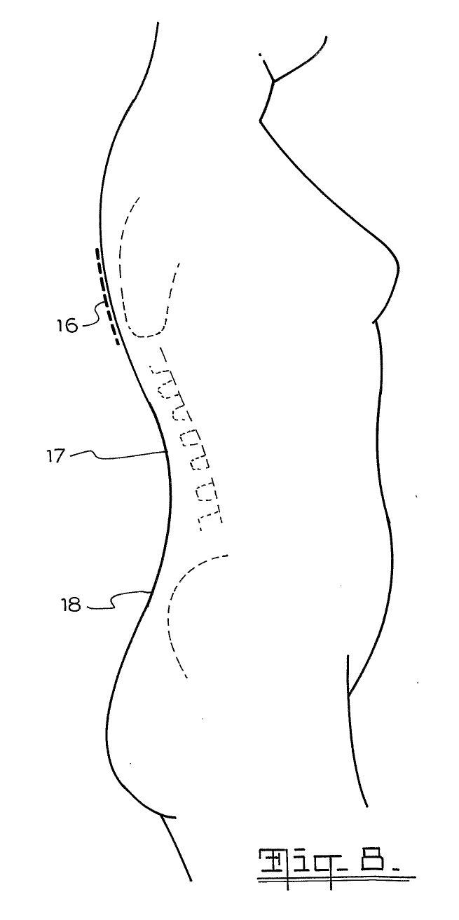

The material of supports 1 and 5 can be an orthopaedic foam, a synthetic rubber or a polymer which is sufficiently rigid to hold the shape of the support without the adhesive backing and so can be applied without buckling. However the material can also have a degree of elasticity allowing minimal flexion from the neutral spinal position and assisting movement back into the correct posture. Accordingly movement into a slouched posture is limited as illustrated in FIG. 7 and tension in the brace serves as a reminder to the wearer to adopt the preferred posture.

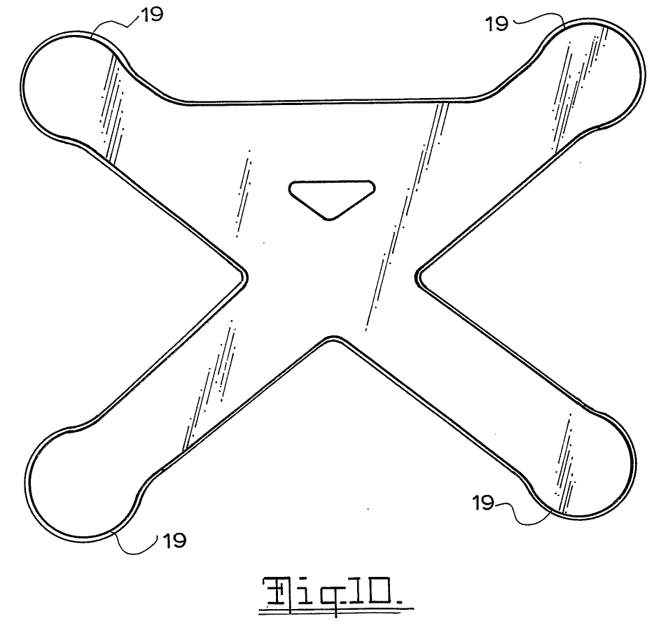

FIG. 8 illustrates the spinal anatomy which provides the rationale for the placement of supports 1 and 5 on the wearer's back. Support 1 is positioned so that the arms of the X cross in the region of the peak of the thoracic kyphosis 16 thus limiting an increase in the latter which would result in an increase in the cervical lordosis as well as in thoracolumbar 17 and lumbar flexion resulting in loss of the neutral spinal curves at all segments of the spine. By positioning the junction of support 1 in the region of the peak of thoracic kyphosis 16, maximum control and maintenance of optimal spinal alignment is achieved.

Furthermore, downward rotation and protraction of the scapulae is prevented due to application of the upper arms of the support to the scapular region.

Correction of posture can be achieved by applying support 1 to prevent thoracolumbar 17 flexion and consequent loss of the neutral spine or by maintaining the lumbar lordosis. The latter is also achieved by positioning support 5 with lower side 6 in the region of the posterior superior iliac spines (PSIS) 18 and the upper-side 7 in the region of the thoracolumbar 17 junction. Accordingly flexion of the lumbar spine between 17 and 18 is reduced and neutral spinal alignment is maintained.

It is important to note that if maintenance of spinal 8 position is achieved at one spinal region, either thoracic 16, thoracolumbar 17 junction or the lumbar spine between 17 and 18, spinal alignment as a whole is generally maintained. The reason for this is that it is difficult to adopt a flexed, kyphotic thoracic posture if the lumbar spine is neutral and vice versa. This is particularly true of the seated position.

Adhesive can be applied directly to the support material during manufacture which results in a single use device. However a double-sided adhesive in the same shape as the support can be used which allows multiple use by replacing the double-sided adhesive each time the support is used. The single use support can also be formed with perforations to allow the skin to breathe and moisture to escape. To achieve the same effect in the multiple use support the adhesive on the wearer's side is applied in a wave or striped form such that only part of the skin directly beneath the support adheres to the support.

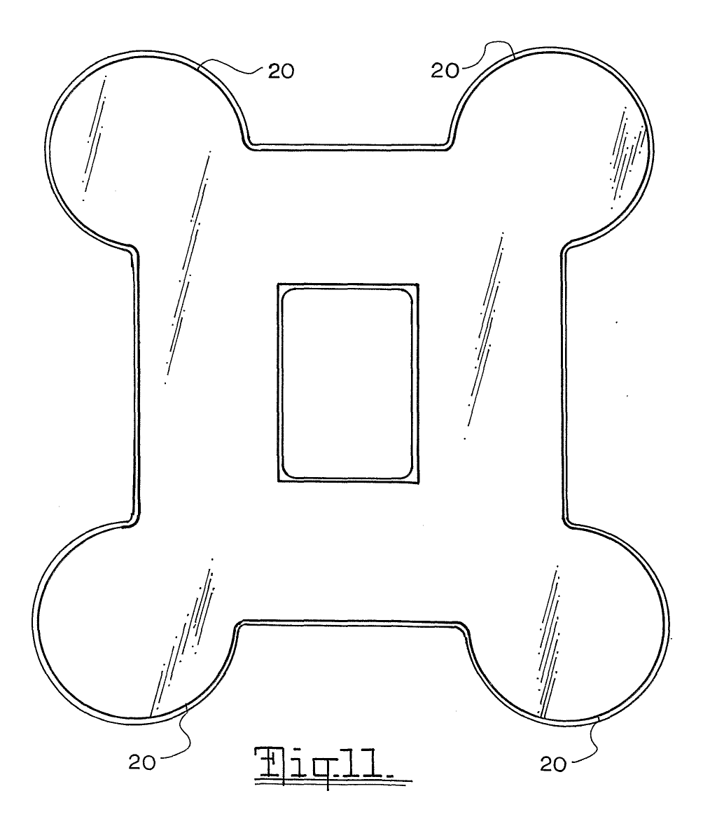

The supports have rounded corners so that there are no sharp edges which are uncomfortable and prone to peeling or lifting from the skin. Alternatively, as shown in FIG. 9, the extremities of the arms of the X shaped support are circular enlargements forming discs which deform if there is tension during application. Tension at the periphery of the support can lead to the formation of traction blisters on the underlying skin.

During application, tension is applied to the arms of the support to achieve the required restriction of movement and to increase the correcting force due to the elasticity of the device material. The circular discs act as an indicator to ensure that no tension is applied to the discs themselves thereby reducing the risk of traction blisters. Similarly, as shown in FIG. 10, the external corners of the square shaped support are circular enlargements forming discs which deform when there is tension in the discs during application.

### Variations

It will be realized that while the foregoing has been given by way of illustrative example of the invention all modifications and variations thereto as would be apparent to persons skilled in the art are deemed to fall within the broad scope and ambit of the invention as herein set forth. Throughout the description and claims of this specification the word “comprise” and variations of that word such as “comprises” and “comprising” are not intended to exclude other additives components, integers or steps.

1. A self adhesive elastic support in the shape of an X which is applied to the wearer's back between the points of the shoulders and the contralateral posterolateral trunk in the region of the lower ribs thereby maintaining body posture in the range of the neutral spine whether the wearer is sitting, standing or lying.

2. The support of claim 1 wherein a cross brace section spans the upper arms of the X shaped support to reinforce the upper arms.

3. A self adhesive elastic support in the form of the sides of a square which is applied to the base of the wearer's back with the lower side in the region of the posterior superior iliac spines and the upper side in the region of the thoracolumbar junction thus reducing flexion of the lumbar spine and preserving neutral spinal alignment.

4. The support of claim 1 in which the self adhesive is applied to the support during manufacture and protected by a peel off strip which is removed prior to application in the case of single use.

5. The support of claim 1 which is provided with double sided adhesive strip attached prior to application to the wearer and can be reused with new double sided adhesive replacing the old.

6. The support of claim 1 which is made from a material which is flexible enough to adapt to the wearer's body shape but rigid enough to maintain correct body posture such as orthopedic foam, rubber or soft plastics.

7. The support of claim 1 which is made from a material which is sufficiently inelastic to limit flexure from the neutral spinal position but assists movement back into the correct posture.

8. The support of claim 1 which is perforated to allow the wearer's skin to breathe and for moisture to escape.

9. The support of claim 1 in which the corners of the X are rounded for optimal adhesion and wearer comfort.

10. The support of claim 1 in which the extremities of the arms of the X are circular enlargements forming discs which deform when there is tension in the discs during application.

11. The support of claim 3 in which the corners of the square are circular enlargements forming discs which deform when there is tension in the discs during application.

12. (canceled)

13. The support of claim 3 in which the self adhesive is applied to the support during manufacture and protected by a peel off strip which is removed prior to application in the case of single use.

14. The support of claim 3 which is provided with double sided adhesive strip attached prior to application to the wearer and can be reused with new double sided adhesive replacing the old.

15. The support of claim 3 which is made from a material which is flexible enough to adapt to the wearer's body shape but rigid enough to maintain correct body posture such as orthopedic foam, rubber or soft plastics.

16. The support of claim 3 which is made from a material which is sufficiently inelastic to limit flexure from the neutral spinal position but assists movement back into the correct posture.

17. The support of claim 3 which is perforated to allow the wearer's skin to breathe and for moisture to escape.
